(19) United States
(12) Patent Application Publication
(10) Pub. No.: US 2005/0177098 A1
(43) Pub. Date: Aug. 11, 2005

(54) Gene injection instrument nozzle

(76) Inventors: Haojan Lin, (US); Chie Song Chen, Taoyuan City (TW)

Correspondence Address: Hung-Chang Lin, 8 Schindler Ct., Silver Spring, MD, 20903, US

(21) Appl. No.: 10/775,400

(22) Filed: Feb. 11, 2004

(51) Int. Cl. A61M 5/30

(52) U.S. Cl. 604/68

(57) ABSTRACT

The nozzle of a gene injection gun is lined inside with a conduit, which has a narrow throat. The throat has a convergent inlet and a divergent outlet. Compressed gas is fed from the inlet, and accelerates to supersonic speed in the throat region. Liquid drug is released to the throat region as droplets, and carried by the accelerated gas flow toward the outlet of the muzzle for injection into a biological body.

### BACKAGROUND OF THE INVENTION

(1) Filed of the Invention

This invention relates to gene injection instrument, particularly the nozzle of a gene injection gun.

(2) Brief Description of Related Art

FIG. 1 shows a side view of a gene injection gun disclosed in U.S. Pat. No. 5,584,807. The gun 10 includes a handle 28, a trigger 30, a feed tube 32 imbedded inside the handle 28. The feed tube serves as a pressure storage chamber for temporary storage of the compressed gas. When the trigger is activated, the compressed gas in the storage chamber is released to the nozzle 33 for injecting the drug solution into a biological body.

At the lower end of the feed tube is a connector 31 for connection to a source of compressed gas (not shown), commonly helium. A valve 34 is used to control the passage of the gas after being released by the trigger 31.

The outlet of the valve is connected to a cartridge holder 36. The cartridge can hold a number of cartridges. Each cartridge can hold a sample of the material to be injected. The cartridge holder can rotate by 360 degrees to place different samples in the conduit 44 leading to the nozzle. The cartridge holder 36 has a number of concave recesses 40. Each recess is used to position a particular cartridge. An elastic convex set plunger 40 is used to plug into the recess to set the position of the particular cartridge and to insure that the correct sample is ejected from the nozzle 44.

The gun body 33 accelerates the drug particles 16. The nozzle 44 has a divergent outlet 46 to shoot out the drug particles 16. The telescopic section 48 of the outlet is adjusted to yield the distance between the cells of the particles.

FIG. 2 shows an expanded view of the prior art nozzle 44. The nozzle has a tubular conduit 442 for accelerating the gas. The cartridge holder 36 holds the drug particles 16, and is placed in the conduit 442. The drawback of this kind of conduit 442 is that the diameter of the conduit is uniform and has excessive length, which creates friction to the gas and prevents the acceleration of the gas.

### SUMMARY OF THE INVENTION

An object of this invention is to increase the instantaneous pressure of gene injection gun and to increase injection speed of the drug particles from the gun. Another object of this invention is to design a throat in the injection gun to increase the instantaneous velocity. Still another object of the present invention is to design a nozzle, which vaporize the drug from a droplet of liquid drug.

These objects are achieved by designing the throat of the nozzle to reduce friction, and to replace the conduit of a conventional gun.

### BRIEF DESCRIPTION OIF THE DRAWINGS

FIG. 1 shows a prior art injection gun for drug.

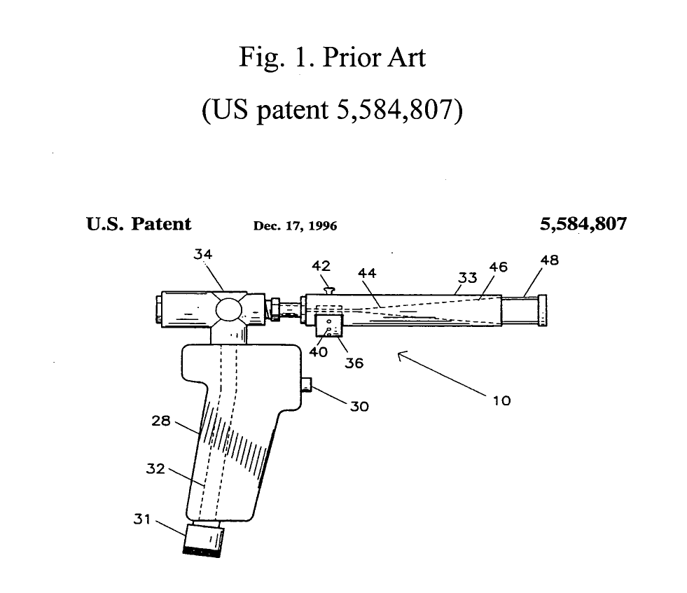

FIG. 2 shows an expanded view of the prior art injection gun.

FIG. 3 shows the throat section of injection gun based on the present invention.

FIG. 4 shows a second embodiment of the throat section of the present invention.

FIG. 5 shows a third embodiment of the throat section of the present invention.

### DETAILED DESCRIPTION OF THE INVENTION

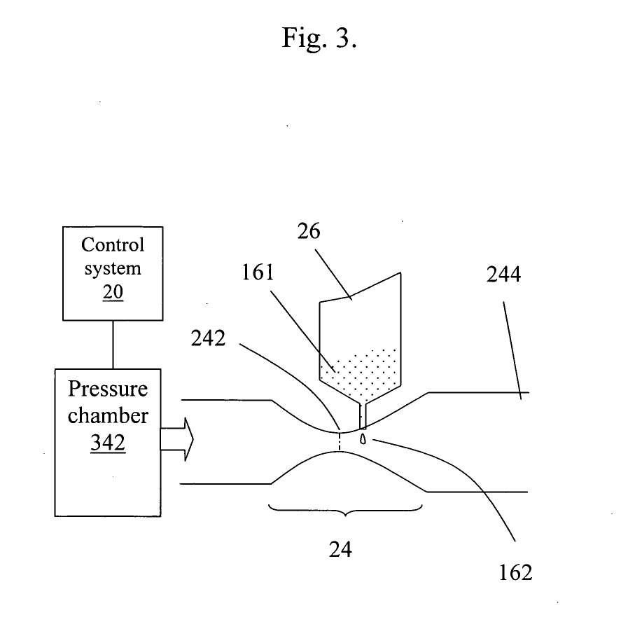

The basic structure of the present invention is shown in FIG. 3. The gene injection gun has a throat section 242. The throat section has a convergent cross section at the incoming end and a divergent cross section at the outgoing end. From the principle of kinetics, the instantaneous pressure of the incoming gas in the convergent section is increased. Then the gas sprays out in accelerated supersonic speed in the divergent section, carrying with it the nucleic acid or protein liquid drug 161 droplets 162. Due to the high speed, the kinetic energy can penetrate through the skin of a biological body and inject the drug into the cell of the body.

For the preparation of the liquid drug 161, the protein or nucleic acid is in a liquid state or pulverized into tiny particles suspended in the a liquid carrier to form a suspended liquid 161.

The tiny drug particles use gold or tungsten particles as carrier. The carriers are coated with the drug, such as nucleic acid or protein. The carriers may also be selected from biological material such as: virus particles, chitosan, collagen, etc. which are imbedded with nucleic or protein to form drug particles for transmission and injection.

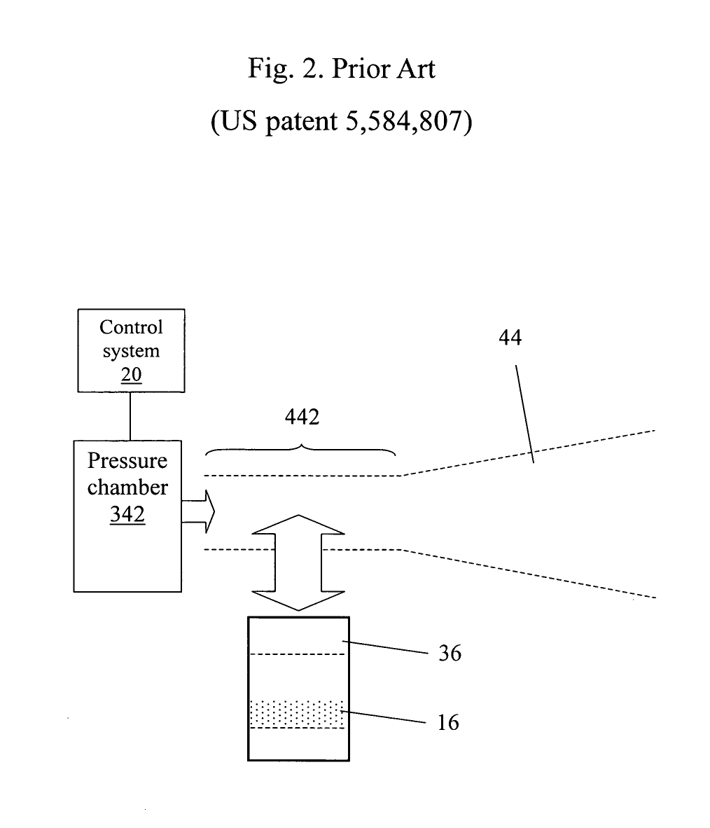

The feature of the present invention lies in the design of the nozzle. In the conventional nozzle design 44 is shown in FIG. 2. The nozzle 24 of the present invention contains a throat section 242 and an outlet 244. The liquid drug droplet 161 is released from a container 26 similar to design of conventional injection tube or injection needle. The liquid drug forms droplet 162 for release into the flow of compressed gas. The highly speed compressed gas flows at supersonic speed in the throat section and injects the drug droplet as mist for injection into a biological body. The drug droplet release container 26 is controlled by a control system 20, such as by means of changing the gas pressure or using a step motor. The form of the droplet 162, which liquid drug 161 in the container 26 is released into the compressed gas flow in the conduit, is controlled by the command from the control system 20, so that the droplet is released drop by drop continuously.

FIG. 3 shows the component parts of the gene injection gun, which utilizes the compressed gas to inject the droplet 162 of the drug 16 into cells of a biological body.

(1) A storage pressure chamber 342 has one end connected to a source of compressed gas for storage.

(2) A nozzle 24 has a narrow throat section 242. The inlet end of the throat 242 has a convergent inlet and is connected to the second end of the storage chamber 342. The outlet of the throat section 242 has a divergent conduit for the compressed gas to eject.

(3) The control system controls the release of the compressed gas to the divergent conduit.

(4) The drug drop container 26 is located near the throat section 242 to supply drug droplet 161 to the divergent conduit. When the compressed is triggered to release, the drug droplet is outputted at supersonic velocity.

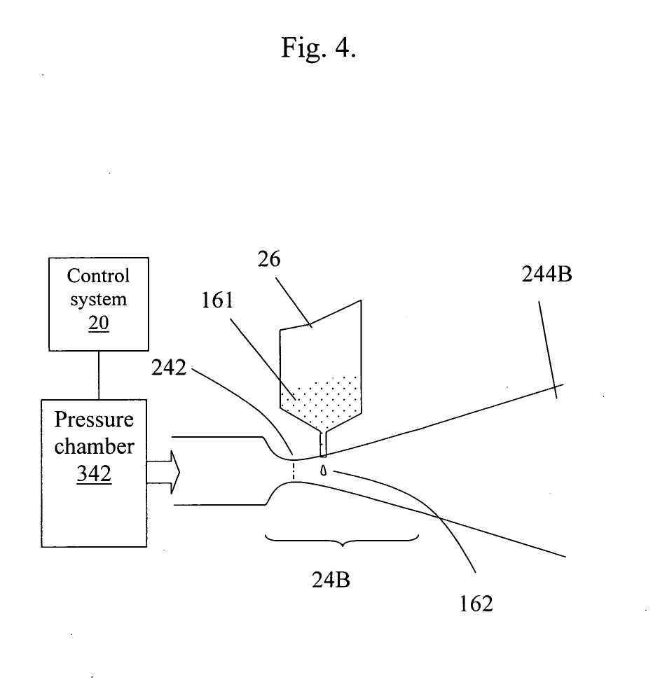

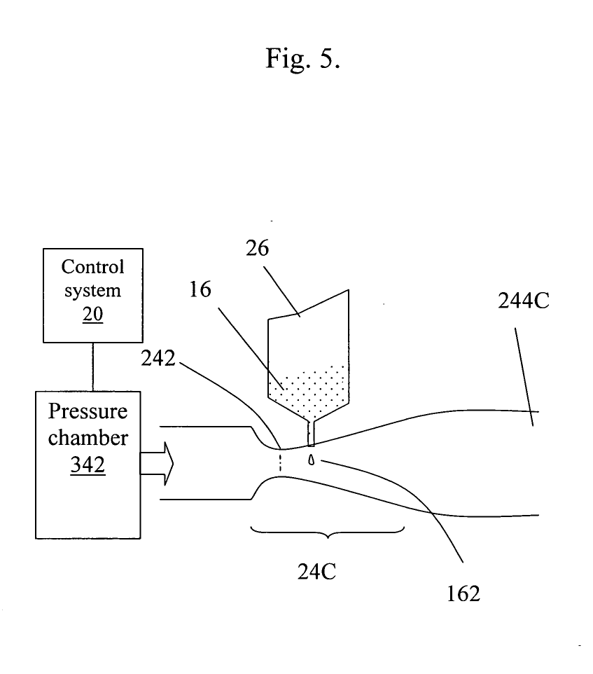

The conduits at the convergent end and the divergent end of the throat section are symmetrical with respect to each other in FIG. 3. The two sections may also be unsymmetrical with respect to the throat section. The conduits at the convergent end would be a constant arc shape in contrast to a conical or bell shape at the divergent end, as shown in FIG. 4 and FIG. 5 respectively.

The drug container may have a needle head for releasing the droplets 162. The outlet of the drug container 26 should be located within 15 mm of the throat region 242.

The throat sections 24, 24B 24C should have a length shorter than 0.822 meter. The drug container can be designed for single release or continuous release. The compress gas for this invention can be carbon dioxide, nitrogen or helium. The compressed air pressure at the inlet of the throat section 242 should be 50-500 psi. The pressure at the outlet of the injection gun should be equal or less one atmospheric pressure (14.7 psi).

While the preferred embodiment of the invention has been described, it will be apparent to those skilled in the art that various modifications can be made to the embodiment without departing from the spirit of the present invention. Such modifications are all within the scope of the present invention.

1. A gene injection instrument for injection drug to a biological body, comprising:
a pressure chamber for storing a gas;
a conduit having one end connected to said pressure chamber and a second end vented for injection into a biological body;
a throat section narrower than said conduit inserted in said conduit having a convergent inlet and a divergent outlet;
a drug container for storing drug in liquid form and located above the throat section with a needle tube to release said drug in liquid form as droplets into the throat section; and
a gas flow from said pressure chamber through said throat section to carry the droplets to the second end of the conduit.

2. The gene injection instrument as described in claim 1, further comprising a control system for controlling the said gas flow.

3. The gene injection instrument as described in claim 1, wherein the gas flow is at supersonic speed.

4. The gene injection instrument as described in claim 1, wherein the pressure in the pressure chamber is set between 50 to 500 psi.

5. The gene injection instrument as described in claim 1, wherein the gas is selected from the group consisting of carbon dioxide, nitrogen and helium.

6. The gene injection instrument as described in claim 1, wherein the needle tube lies within 15 mm of the center of the throat section.

7. The gene injection instrument as described in claim 1, wherein the convergent inlet and the divergent outlet are symmetrical.

8. The gene injection instrument as described in claim 1, wherein convergent inlet and the divergent output are unsymmetrical.

9. The gene injection instrument as described in claim 2, wherein the control system controls the release of the droplets.

10. The gene injection instrument as described in claim 9, wherein the control system controls by means of changing the pressure of the pressure chamber.

11. The gene injection instrument as described in claim 9, wherein the control system controls by means of a stepping motor.
